(19) United States
(12) Patent Application Publication
(10) Pub. No.: US 2005/0058744 A1
(43) Pub. Date: Mar. 17, 2005

(54) Edible compositions capable of removing oral biofilm

(75) Inventors: Doron Steinberg, Jerusalem (IL); Alexander Kamyshny, Jerusalem (IL); Shlomo Magdassi, Jerusalem (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(73) Assignee: YISSUM RESEARCH DEVELOPMENT COMPANY OF THE HEBREW UNIVERSITY OF JERUSALEM, Jerusalem (IL)

(21) Appl. No.: 10/884,631

(22) Filed: Jul. 1, 2004

Related U.S. Application Data
(63) Continuation of application No. PCT/IL03/00001, filed on Jan. 1, 2003.

(51) Int. Cl. A23G 3/30

(52) U.S. Cl. 426/3

(57) ABSTRACT

An edible and/or chewable article of manufacture containing at least one food grade substance having adsorption affinity towards at least one dental plaque (biofilm) constituent and capable of reducing and/or removing the oral biofilm while present in the mouth. Particular articles of manufacture are chewing gums, sweets, candies, candy, and other nutritional bars, ice creams, chocolates, confectionery and bakery/pastry products, honey, dairy products and beverages, and oral hygiene products such as tooth pastes, oral gels and mouthwashes. A chewing gum having a conventional gum base and at least one food grade active substance having adsorption affinity towards at least one dental plaque (biofilm) constituent (bacteria and proteins and bacterial cell-free enzymes) and capable of reducing and/or removing the oral biofilm while present in the mouth. Active substances include polysaccharides and non-toxic salts thereof, such as alginates, chitosan, carboxymethylcellulose, agar and carrageenan, inorganic substances such as silica, hydroxyapatite and calcium carbonate and proteins, particularly gelatin and lectin. The article of manufacture removes and/or for prevents or reduces dental plaque (biofilm), and controlling oral, dental and periodontal diseases.

### CROSS REFERENCE TO RELATED APPLICATION

This application is a continuation of International Patent Application No. PCT/IL03/00001 filed Jan. 1, 2003, the contents of which are here incorporated in their entirety. Applicant claims the benefit of 35 USC 120.

### BACKGROUND OF THE INVENTION

1. Field of the Invention

The present invention relates to the field of prevention and removal of dental plaque biofilm by means of substances which are capable of adsorbing oral biofilm constituents, including bacteria, which are incorporated into edibles, particularly into chewing gum. More specifically, the present invention provides modified chewing gum compositions capable of effectively adsorbing and removing oral bacteria and other constituents of the dental biofilm. This adhesion/adsorption property contributes to substantial reduction in plaque formation and accumulation, resulting in reduced risk for dental and oral diseases.

2. Prior Art

Throughout this application various publications are indicated by Arab numerals in parentheses. A comprehensive list of these publications appears at the end of the description, immediately preceding the claims. All of these publications, including publications referred to therein, are fully incorporated herein by reference.

Dental diseases such as tooth decay, gingivitis, periodontal diseases, and oral fungal infections are recognized as one of the major worldwide public health problems (1). Furthermore, severe economic problems are associated with dental diseases (2).

These oral infectious diseases are associated with the development of the dental plaque biofilm. Dental caries (tooth decay) is found most often in children and youths. Gingivitis occurs in children as well as in adults, while periodontal diseases progress with advancing age.

The ultimate outcome of these dental diseases is destruction of the tooth enamel, dentine cementum and the supporting periodontal tissues. Tooth loss, pain, discomfort, cosmetic considerations, impaired speech, chewing and eating difficulties are complications associated with dental diseases. Financial considerations of dental treatment are yet another aspect of dental diseases. Unlike other infectious diseases, which have been controlled, the susceptibility to oral diseases has not declined. It is evident that without proper dental care, the risk of recurrence of dental disease is extremely high at all age, race and sex groups, at all socioeconomic levels. Therefore, it is of utmost importance to treat, control and prevent dental diseases.

Primary dental care begins at home. The practice of regular oral hygiene, such as tooth brushing, mouth rinsing and dental flossing plays a vital role in maintaining healthy teeth and gums. Nevertheless, removal of the dental biofilm by tooth brushing is effective but is limited to very specific times.

Professional treatments by dental practitioners are additional requirements for healthy oral maintenance. However, additional means of oral hygiene as chewing gums, which are actively influencing the virulent factors of the oral diseases can attribute to the ongoing level of prevention and treatment.

Dental Biofilm with Relation to Dental Diseases

The dental biofilm is a deposit of proteins, bacterial cell-free enzymes and bacteria embedded in exopolysaccharides, which adhere firmly to the tooth surfaces. Oral biofilms harboring pathogenic bacteria and other virulent factors are associated with oral diseases, such as tooth decay gingivitis and periodontal diseases (3).

Numerous types of hard and soft surfaces are part of the oral cavity. Diversity is the key feature of the oral biofilms formed in the oral cavity. Among the hard, non-shedding surfaces are enamel, restorative materials, implants, prosthetic and orthodontic appliances. All of these differ in their chemical and physical surface properties and in their surface topography.

Oral bacteria, including cariogenic bacteria, are part of the commensal flora of the oral cavity. Changes in the oral environment, mainly in diet, can cause the existing microflora to become potentially pathogenic. Among the bacteria associated with dental caries are Streptococcus mutans (S. mutans), Streptococcus sobrinus (S. sobrinus), and lactobacilli. These cariogenic bacteria are aciduric as well as acidogenic types of bacteria. Actinomyces viscosus (A. viscosus) and Streptococcus sanguis (S. sanguis) are additional oral bacteria whose ecological niche is the dental plaque (4).

Periodontal diseases are inflammatory responses in which the structural support of the tooth is destroyed. The initial stage of periodontal diseases is associated with the formation of the supra-gingival dental biofilm. The clinical manifestation resulting from the destruction of the periodontal ligaments is the formation of a deeper space between the root surface and the opposing periodontal tissue. This crevice is termed a periodontal pocket. Periodontal diseases are characterized by a dramatic change of the microflora surrounding the tooth; the number of gram-negative bacteria within a periodontal pocket can increase to 70% of the total flora, most of it anaerobic bacteria. There are numerous types of bacteria affiliated with periodontal diseases, for example: Porphyromonas gingivalis, Bacteriodes melaninogenicus, Fusobacterium, Capnocytophaga and spirochetes. These bacteria, as well as others, play an important role in the pathogenicity of the diseases. Collagenase and other enzymes originating from these bacteria can destroy the connective tissue of ligaments in the periodontum. Toxins excreted by the periopathogenic bacteria contribute to the progress of periodontal diseases by evoking an inflammatory host response.

Bacterial Adhesion Mechanisms (Biofilm Formation)

Microbial cells are capable of attachment to almost any surface submerged in an aqueous environment—a phenomenon known as microbial adhesion. Colonization and proliferation of the bacterial cells on a surface forms a biofilm.

Adhesion of microorganisms on a surface is involved in certain diseases of humans and animals, in dental plaque formation, in industrial processes, in fouling of man-made surfaces, in syntrophic and other community interactions between microorganisms, and in the activity and survival of microorganisms in natural habitats (5). Microorganisms are living colloidal systems. Therefore, combining the knowledge of colloid, interface and microbiology sciences seems to be promising in understanding the effect of biofilm.

Microbial adhesion may be roughly divided into two types: nonspecific and specific. Initial (nonspecific) reversible adhesion is mainly a physicochemical process (5). A number of classes of interaction may be involved in nonspecific adhesion: Van der Waals forces, dipolar, electrostatic, hydrogen bonds, hydrophobic interactions. The number and strength of these interactions vary considerably from system to system, depending on the type of the surface and microbe involved. For instance, part or the entire external surface of some bacterial cells is hydrophobic, and the adhesion of some bacterial strains onto hydrophobic sulphated polystyrene should correlate with bacterial hydrophobicity. The contact angle is a relative measure for representing the degree of hydrophobicity of a surface, which in most cases shows a correlation with the surface Gibbs energy (the surface Gibbs energy decreases with an increase in hydrophobicity).

The majority of bacteria are negatively charged (the negative charge on the bacteria surface may originate from the cell wall and/or from the extracellular polymer layer) and will electrostatically attract to positively charged surfaces and repel from negatively charged surfaces. However, since positively charged solid surfaces are very rare in nature, a repulsion energy barrier for microbial adsorption does exist. The repulsion between a negative surface and bacterial cell may well be overcome by the polymer-surface interaction, i.e. many weak polymer-surface contacts may provide sufficient energy to overcome an electrical repulsion in the adsorbed state. An important contribution to adhesion may come from the extracellular enzymes often associated with the bacteria. Enzymes contain both negatively and positively charged groups or sites, and other groups capable of promoting adhesion through various mechanisms such as hydrogen bonding. Therefore, the polysaccharides, the exocellular enzymes and proteins may provide binding/adsorption sites, for example, the positive groups can form strong bonds to negative sites on the surface (6).

The other type of adhesion, specific adhesion, is determined by the presence of special structures on the surfaces of the bacterial cell, such as fimbriae (filamentous structures of uniform diameter which function as adhesive organelles), pili (organelles involved in conjugation and DNA transport between cells), and other appendages or polymer molecules expanding into surrounding environment (7). Such specific organelles and molecules are named “adhesins”. These molecules may be, for instance, acidic polysaccharides or glycoproteins (8). Adhesins recognize specific molecules, binding sites (mainly, carbohydrate derivatives), located on the surface, thereby anchoring the bacterium to the surface (8-10). Interaction between adhesins of microbial cells and receptors of the surface is determined by lock-and-key mechanism.

Proteins such as lectins, which are carbohydrate-binding proteins, may serve as adhesins (8, 10). Lectin-like, binding sites-mediated interactions have been considered important even for bacterial adhesion to inert surfaces such as hydroxyapatite. The possibility of a specific hydrophobic interaction involving specific recognition between a hydrophobic moiety on the bacterial cell surface and a substratum receptor also should be taken into account. These examples may be relevant for inert surfaces in nature and are interesting to bear in mind for discussions on short-range forces and the general correlation between degree of hydrophobicity and adhesion that exists in a wide range of systems (8, 10).

Experimental evidence suggests that the receptor moiety which is bound by the adhesin is a relatively small molecule, perhaps no larger than a mono- or disaccharide (α-D-mannosyl, β-D-mannosyl, D-mannoside, α-D-galactosyl, β-D-galactosyl, neuraminic acid, L-fucosyl). This evidence is supported by the fact that sugars (α-methyl-D-mannoside, β-D-mannoside, D-mannose, α-D-galactoside, lactose, L-fucoside, neuraminic acid, chemically degraded glycoproteins) can specifically inhibit adhesion (10). Lectins can also inhibit adhesion by competing for the adhesin receptors.

The strategy of antimicrobial protection is based on the blocking the interaction of bacterial cell with binding sites on the surface. There are several approaches to antimicrobial protection.

Adding highly adhesive material (substance), which competes with the bacterial adhesion forces to the solid surface (tooth), will result in detachment of bacteria from the surface.

Cations can interact with the negative charged groups of a surface of the bacteria, thus assist in the adhesion of bacteria to surfaces. For instance, polycationic polymers, such as chitosan, poly-L-lysine and lysozyme, are effective in microbial cell binding by electrostatic interactions, i.e. these substances can compete for microbial adhesion. Chitosan was also shown to be effective as antimicrobial agent against listeriae, salmonellae and yeast adhered to stainless steel disks (11).

Proteins as fibronectin and laminin (12, 13) were shown to promote adhesion, therefore they can also serve as competitors in microbial adsorption.

Attachment may be induced by substances, which block the interaction between the bacterial adhesins and the target surface receptor. These include: the binding site analogs, blocking the adhesin binding sites, antibodies against binding site of adhesin or binding sites, antibiotics and other antibacterial agents, which block the synthesis of bacterial adhesins or interfere with bacterial attachment (14).

The influence of proteins on adhesion has been studied (15) and it has been reported that the adhesion to glass is stimulated by casein and gelatin and decreased by protamine and bovine serum albumin (BSA).

Loosening the network of bacteria in the biofilm while implementing a stronger attraction force will also affect the biofilm.

Poloxamer 407 was shown to have an anti-adhesive effect on bacterial adherence to polymethylmethacrylate and enhanced the susceptibility of bacteria to antibiotics (16). Condensed tannins and methylcellulose were found to prevent microbial attachment and subsequent digestion of cellulose (17).

Some investigators have attempted to remove adherent bacteria through use of enzymes, which degrade their bridging polymers (8, 18). An example is the application of glucan hydrolases, which attack the extracellular glucans, which promote accumulation of Streptococcus mutans cells on teeth (8).

Some proteins will also reduce biofilm formation. Adhesion of a marine Pseudomonas to polystyrene decreased due to the presence of BSA, gelatin, fibrinogen, protamine and pepsin (9). When free proteins were present during the attachment, the strongest influence on adhesion was observed, presumably due to protein adsorption on both, the bacterial and polystyrene surface. Pretreatment of the polystyrene surface with proteins also led to a reduction of the adhesion (except pretreatment with protamine), whereas the pretreatment of bacteria resulted in decreased adhesion for BSA-treated cells only. Probably, the adsorption of proteins had a greater influence on the hydrophobicity of the substrate surface than on the surface of bacteria. The fact that proteins not only influence the hydrophobic but also electrostatic interaction may be inferred from the observation that the basic proteins protamine and histone (which are positively charged at pH 7) have no influence on adhesion when adsorbed on polystyrene (9), presumably a decreased Van der Waals attraction is balanced by a decreased electrostatic repulsion.

In addition, there are several patents claiming methods for inhibiting the microbial adhesion on surfaces. Wright et al. (19-21) propose to use combination of alkylsulfosuccinate surfactant with alkyl chain length from 5 to 13 carbon atoms and polyoxyethylene-polyoxypropylene block copolymer surfactant. Donald et al. (22) used ethoxylated nonionic surfactant which is a block copolymer of repeating ethylene oxide and repeating propylene oxide units for inhibiting the microbial colonization of a hydrophobic surface.

Adsorbed bacteria can in many cases be removed by periodate treatment (15). The periodate sensitivity of the polymer suggests it is a polysaccharide or glycoprotein.

In spite of accumulated data regarding the adsorption of bacterial cells onto a material surface, there is no data available relating to the use of chewing gum for adsorbing oral bacteria, thus eliminating or reducing the formation of dental plaque biofilm, which leads to development of oral diseases.

Consequently, it is the object of present invention to provide edibles, and particularly a chewing gum, capable of adsorbing oral bacteria and removing the bacteria which are bound to the teeth through the biofilm, while the chewing continues, or while the edible product, such as candy, is present in the mouth. It is a further object of present invention to provide such edibles and/or chewing gum and/or dental hygiene products such as tooth pastes that are capable of reducing dental plaque biofilm formation. It is yet a further object of present invention to prevent or reduce oral and dental diseases by means of chewing an edible substance, particularly a chewing gum, which is capable of adsorbing oral bacteria. It is yet a further object of present invention to provide a process for preparing a chewing gum capable of adsorbing oral bacteria. It is yet a further object of present invention to provide an edible substance, preferably chewing gum, with can release the said active agent (the adhesive substance) in a sustained manner, thus prolonging the biofilm adsorption effect.

Conventional chewing gum contains the gum base (which is a mixture of polymers), flavoring agents and different additives, particularly additives which provide for an improved chewing action, such as additives affording non-stickiness. However, conventional chewing gums, as shown in the following examples, do not have the ability to cause desorption of the bacteria and the biofilm off surfaces.

### SUMMARY OF THE INVENTION

The present invention provides compositions of chewing gum, which are capable of significantly adsorbing oral bacteria. The new chewing gum contains substances which induce high adhesion properties, thus attracting constituents from the biofilm onto the chewing gum. Alternatively or additionally, the chewing gum of the invention can contain and release substances which will reduce bacterial adhesion to oral cavity surfaces, causing the bacteria to adsorb to a more strongly attracting surface—the chewing gum. The chewing gum, therefore, provides for removal of the adsorbed oral bacteria while the chewing gum is chewed, as a result of the chewing action, which is combined with the bacteria-binding ability of the chewing gum due to the specific added components.

The invention relates to an edible and/or chewable article of manufacture containing at least one food grade substance having adsorption affinity towards at least one dental plaque (biofilm) constituent and capable of reducing and/or removing at least part of the oral biofilm while present in the mouth.

An article of manufacture in accordance with the invention may be selected from the group consisting of chewing gums, sweets, candies, candy- and other nutritional bars, ice creams, confectionery and bakery/pastry products, honey, dairy products and beverages. Alternatively, the article of manufacture may be selected from the group consisting of tooth pastes, oral gels and mouthwashes.

In a preferred embodiment, the invention relates to a chewing gum, wherein said chewing gum comprises a conventional gum base and at least one food grade active substance having adsorption affinity towards at least one dental plaque (biofilm) constituent and capable of reducing and/or removing the oral biofilm while present in the mouth.

Preferably, the chewing gum of the invention has affinity towards said at least plaque constituent higher than that of the gum base comprised therein.

The dental plaque (biofilm) constituent is a bacterium of the oral cavity of at least one species, particularly, but not limited to bacteria selected from the group consisting of Streptococcus mutans, Streptococcus sobrinus, Streptococcus salivarius and Actinomyces viscosus.

Alternatively, the dental plaque constituent is selected from the group consisting of host and bacterial proteins and cell-free enzymes, for example glucosyl tranferase.

The active substance comprised in the article of the invention, particularly a chewing gum, is preferably a polysaccharide or a non-toxic salt thereof, particularly any one of alginate, preferably sodium alginate, chitosan, carboxymethylcellulose, agar and carrageenan.

Alternatively, the active substance is an inorganic substance, particularly any one of silica, hydroxyapatite and calcium carbonate.

Still alternatively, the active substance is a protein, preferably a gelatin or lectin.

The active substance may also comprise a mixture of at least two substances selected from polysaccharides, proteins and inorganic substances. In particular, the polysaccharides may be selected from the group consisting of alginates, preferably sodium alginate, chitosan, carboxymethylcellulose, agar and carrageenan, the proteins may be selected from lectins and gelatins, preferably gelatin A, and the inorganic materials may be selected from the group consisting of silica, hydroxyapatite and calcium carbonate.

The article of manufacture in accordance with the invention, particularly chewing gum, are particularly intended for removing and/or for preventing or reducing dental plaque (biofilm).

The article of manufacture in accordance with the invention, particularly chewing gum, may optionally further comprise at least one food grade additive, particularly colouring agents, flavouring agents, breath freshening agents, pH controlling agents and preserving agents.

In a specific embodiment, the invention relates to a chewing gum comprising chitosan as the active substance and citric acid as a pH controlling agent.

The chewing gum according to the invention may be in unit form, preferably in the form of a bar, drop, sphere or stick.

In another aspect, the invention relates to the use of an active substance which has adsorption affinity towards dental plaque (biofilm) constituents, in the preparation of an edible and/or chewable article of manufacture adapted for removing dental plaque (biofilm) and/or for preventing or reducing dental plaque.

Also in this aspect, the article of manufacture may be selected from the group consisting of chewing gums, sweets, candies, candy- and other nutritional bars, ice creams, confectionery and bakery/pastry products, chocolates, honey, dairy products and beverages, or alternatively may be any one an oral hygiene product such as a tooth paste, an oral gel and a mouthwash.

In a preferred embodiment, the use of the invention is directed at the manufacture of a chewing gum which contains a conventional chewing gum base and an active food grade substance which has adsorption affinity towards dental plaque (biofilm) constituents, wherein the adsorption affinity of said chewing gum towards dental plaque (biofilm) constituents is higher than the affinity of said gum base.

The preferred characteristics of the articles of manufacture in accordance with the invention apply to the articles of manufactures for use in accordance with the invention.

All the above and other characteristics and advantages of the invention will be further understood through the following illustrative and non-limitative description of preferred embodiments thereof, with reference to the appended drawings.

### BRIEF DESCRIPTION OF THE DRAWINGS

FIG. 1: Adhesion of Strep. mutans to chewing gums containing polysaccharides and poly-saccharide derivatives.

FIG. 2: Adhesion of Strep. mutans to chewing gums containing inorganic substances.

FIG. 3: Adhesion of Strep. mutans to chewing gums containing proteins.

### DESCRIPTION OF THE INVENTION

The adhesive-containing substance of the invention, particularly the chewing gum of present invention, reduces the adhesion of oral bacteria to hard surfaces in the oral cavity (e.g. restorations, enamel, cement, dentine, implants, dentures, orthodontic appliances) and thus fully or partially eliminates one of the main events leading to development of an oral disease. Decreasing the extent of bacterial cell adhesion onto oral surfaces would result in reducing the formation of dental plaque biofilm. Decreasing dental plaque leads to prevention of tooth decay, calculus, gingivitis and periodontal diseases. Thus, eliminating and removing bacterial depositions in the oral cavity are of major importance in combating oral diseases.

Chewing gum and sweets, as well as other edible, are very common among both adult and young population. The inventors have aimed at edible compositions which can remove adsorbed oral bacteria, induce a special oral cleaning effect while the edible product is present in the mouth and/or while the chewing continues. Thus, combining ingredients which have high affinity towards biofilm components into the food product, with the mechanical chewing action, provides a novel way of removing oral bacteria. More particularly, the present invention provides a modified chewing gum comprising or supplemented with unique additives which have highly adhesive properties towards dental biofilm components. In particular, these additives possess strong affinity towards bacteria, and probably also towards cell-free enzymes and proteins, which are integral part of the dental plaque biofilm. The modified chewing gum and edibles of the present invention remove these components from the biofilm, resulting in disintegration of the biofilm and/or prevention of its formation. Chewing the gum, supplemented with these adhesive ingredients, will reduce plaque. Consequently, the gum of the present invention, having high affinity towards the constituents of the biofilm, will disintegrate the plaque, and after a while, the gum, together with the adsorbed bacteria and/or other biofilm constituents is disposed of. In case of an edible product according to the invention, the bacteria are adsorbed by the chewed mass, and may then be swallowed therewith and thus removed from the oral cavity. Since the oral bacteria are not pathogenic, swallowing them would cause no damage to the consumer. This mode of action, by applying the chewing gum or edibles of the present invention, has some profound advantages, such as simplicity of use, high compliance and low cost. Chewing gums are in common use by adults and children, and the use of the chewing gum of present invention may be readily accepted by the public, thus accompanied by high compliance. In addition, the use of chewing gum does not interfere with other oral hygiene regimes; rather, the chewing gum of the present invention has supplementary beneficiary effects in preventing dental diseases.

The application of the present invention, namely the addition of adhesive constituents, can be expanded to food products (edibles) such as sweets, candies, candy and other nutritional bars, ice creams, confectionery and bakery/pastry products, honey, dairy products and beverages. Furthermore, the addition of adhesive constituents according to the present invention can also be expanded to oral hygienic products such as tooth pastes, mouthwashes and gels. The uses of the special adhesives of the invention can be adapted for, e.g. pharmaceutical, medical or veterinary applications, for various oral and dental applications.

The chewing gum composition of the present invention comprises a conventional chewing gum base. By the term “conventional chewing gum base” as used herein is meant a mixture of polymers and sweeteners, which provides good organoleptic properties whilst the chewing gum is chewed. In general, chewing gum bases are the chewy, water-insoluble components of chewing gum and bubble gum. Structurally, they are complex mixtures of food grade elastomers, resin plasticizers, minerals, waxes, lipids and emulsifiers. The elastomers include natural gums (such as crown gum, gutta hang kang), polyisobutylene, styrene-butadiene rubber, etc.

More specifically, the gum base may generally comprises elastomers (8-13%), resins (25-35%), plasticizers (10-20%), water insoluble adjuvants (30-40%), antioxidants (0.09%) and BHT (butylated hydroxytoluene.)

Specific constituents of the gum base may be polyisobutylene, isobutylene-isoprene copolymer, fully refined microcrystalline wax, polyvinyl acetate, glycerol ester gum rosins, polyterpene resin, acetylated monoglycerides, BHT, lecithins, glycerol monostearate, hydrogenated vegetable oils and talcum.

A conventional chewing gum may generally comprise carbohydrates/sugar alcohols (sorbitol, mannitol, xylitol, isomaltitol, maltitol syrup) (60-70%), gum base (20-30%), flavors and sweeteners (such as aspartame, acesulfam K), stabilizers (such as gum Arabic) and food colorants.

The chewing gum composition comprises as an active ingredient a substance, which has a higher adsorption affinity to dental plaque constituents than the conventional gum base. In particular embodiments, the adhesive active ingredient is selected from the group consisting of polysaccharides and non-toxic salts thereof, preferably alginates and particularly sodium alginate, chitosan, carboxymethylcellulose, agar and carrageenan. Alternatively, the active ingredient may be an inorganic substance, such as silica, hydroxyapatite and calcium carbonate. In other embodiments, the active ingredient may be a protein, such as gelatin. The chewing gum of the invention may also consist of a mixture of at least two of said active ingredients. When combining two or more active substances, or one active substance and additive/s, their respective quantities and specific combination may be designed so as to adjust the strength of adsorption to good organoleptic properties. Thus, for example, CMC and sodium alginate can be added together to the gum base, to ensure that the chewing gum is both highly adsorptive and well organoleptic.

The chewing gum of the invention contains an effective amount of the active ingredient. By the term “effective amount” is meant a concentration of active ingredient that would cause stronger adhesion of the biofilm constituent to the chewing gum than to oral cavity surface, and/or cause release of the biofilm constituents from the plaque and their adsorption by the chewing gum. Preferably, the chewing gum of the invention contains the active ingredient at a concentration of from 0.5 % w/w to 30 % w/w, more preferably from 10% w/w to 20% w/w and particularly from 15% w/w to 17% w/w.

A typical conventional chewing gum may contain, for example: gum base 19.4%, corn syrup 19.8%, sugar 59.7%, flavor 0.6%. Clearly this is but an example, and variations are possible, as known to the man of skill in the art. Thus, the active ingredient may be incorporated into various types of chewing gum, such as low and high moisture, containing or not containing wax, sugar-sweetened or sugar-free, etc.

In addition, the chewing gum composition of the invention may comprise edible additives such as conventional coloring and flavoring agents, preserving agents, pH controlling agents, breath freshening agents and the like.

As shown in the following Examples, a chewing gum according to the invention may contain chitosan as the active agent, together with citric acid, which enhances the bacteria-adsorptive properties.

In addition, the chewing gum of the invention may contain additional dental agents such as antibacterial agents, teeth-bleaching agents, fluoridating agents and abrasives.

As mentioned above, the chewing gum of the invention can release the active agent (the adhesive substance) in a sustained manner, thus prolonging the biofilm adsorption effect. The continued mastication prolongs the contact of the active ingredient with the oral biofilm. This effect can be enhanced by various techniques, such as encapsulating the active ingredient, for providing an even longer effective application time.

The chewing gum composition is preferably to be marketed in unit form, particularly conventional bar and spherical forms.

The invention further relates to the use of the said active ingredient in the production of other food grade articles of manufacture capable of enhancing oral hygiene and reducing dental plaque. Such articles of manufacture can be, in addition to chewing gum, e.g. sweets, candies, candy- and other nutritional bars, ice creams, confectionery and bakery/pastry products, chocolates, honey, dairy products and beverages. Other oral hygiene products such as are tooth pastes and mouthwashes gels.

In the use of the present invention, the active ingredient, or mixture of active ingredients, has adsorption affinity towards dental plaque (biofilm) constituents higher than the affinity of said bacteria to oral cavity surfaces. All of the above-mentioned active ingredients can be used in the preparation of the other articles of manufacture.

The chewing gum (and other products) of the invention is particularly useful in promoting oral hygiene and preventing dental and periodontal diseases. This is achieved by masticating the chewing gum. As the gum is chewed, the active ingredient is brought into continuing contact with oral bacteria and other biofilm constituents, whether adhered to oral cavity surfaces or present in the saliva, and adsorbs them. After a desired period of time, the chewing gum, with undesired biofilm constituents adhered thereto is dispensed with.

The terms chewing gum and chewing gum compositions are used herein interchangingly.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

Materials and Methods

1. Chewing Gum Preparation

6.25 g of the chewing gum base (Helen, Helen-T from Gum Base Co.) was heated in a microwave oven up to the paste state and mixed with the indicated amount of an additive. Then chewing gum base discs with diameter ˜5-6 mm and thickness of ˜2 mm were formed and allowed to harden.

2. Bacterial Adhesion Assays

The chewing gum discs were cut into pieces and washed twice with saline, and then incubated with 1 ml of radioactive bacteria (prepared as described below). After 15 minutes of incubation at 370 C, the chewing gum discs were washed to remove loosely adhered bacteria. The chewing gum discs were then transferred into a scintillation fluid and the amount of radioactive counts was measured in a scintillation counter. Data presented as percent of adhesion compared to placebo (gum-base).

3. Radioactive Bacteria

Four representative cariogenic bacteria were tested as follows:

Streptococcus mutans, Streptococcus sobrinus, Streptococcus salivarius and Actinomyces viscosus. These bacteria were radioactively labeled to facilitate assessing bacterial adhesion in the following assays. The above bacteria were grown in brain heart infusion supplemented with 3H-Thymidine. After overnight incubation at 370 C at 5% CO2, the bacteria were washed three times with PBS and the concentration was adjusted to optical density of 1.2 at 540 nm.

### Example 1

6.25 g of chewing gum base were heated in a microwave oven up to the paste state and were mechanically mixed with 1.25 g of a powder or granules of a polysaccharide or polysaccharide derivative. The resulting matrix is composed of the gum base and the polysaccharide particles dispersed within it. Then the chewing gum/polysaccharide matrix was shaped into discs with diameter of ˜5-6 mm and thickness of ˜2 mm, which were allowed to harden upon cooling. The following polysaccharides were tested: chitosan (Amercol, Kytamer L), sodium alginate (Sigma Chemical Co.), calcium alginate (prepared as precipitate after mixing 100 ml of 1% sodium alginate and 100 ml of 2% CaCl2 solutions), sodium salt of high viscosity carboxymethylcellulose, CMC (Sigma Chemical Co.), ethylcellulose (Sigma Chemical Co.), methylcellulose (Viscotran, Aadopri-20), microcrystalline cellulose (Avicel, FMC), guar gum (Sigma Chemical Co.), xanthane (Rhodia, Type 200), locust bean gum (Sigma Chemical Co.), dextran, M.W. 190 kDa and dextran M.W. 9.3 kDa (both Sigma Chemical Co.), citrus pectin potassium salt (Sigma Chemical Co.), agars (Difco Laboratories (USA) and Fluka+), starch (Merck), carrageenan (Sigma Chemical Co.).

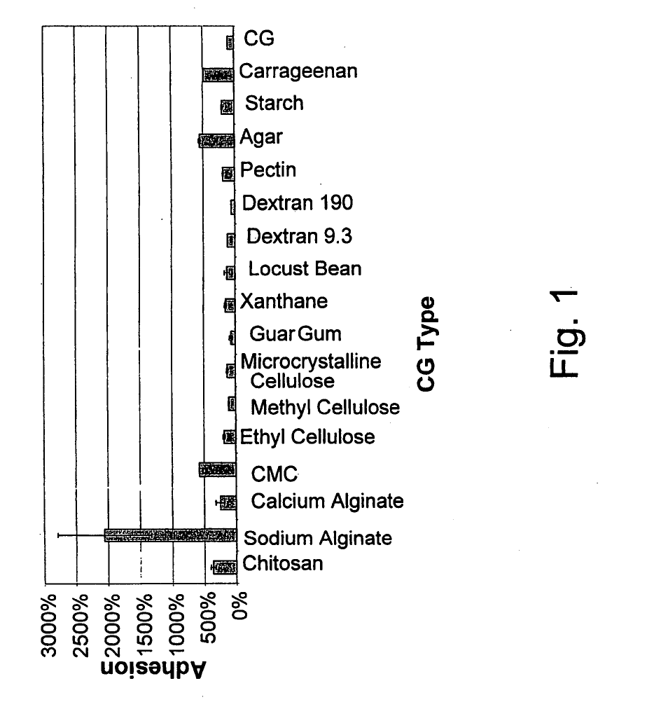

The discs were then incubated with the 3H-labeled bacteria, as described above. The results shown in FIG. 1 demonstrate that only a few of the additives significantly improved the bacterial adhesion to the gum base. Specifically, chewing gum base containing sodium alginate exhibited the best binding properties for the bacteria Strep. mutans, average of 2000% compared to the gum base without the alginate (control=100% adhesion). Other polysaccharides, like carrageenan, agar, CMC and chitosan also exhibited higher adsorption than the control chewing gum base (CG), but less than the alginate (500%, 510%, 600%, 400%, respectively).

### Example 2

6.25 g of chewing gum base were heated in a microwave oven up to the paste state and mixed with 0.625-1.25 g of inorganic substances. Then chewing gum discs with a diameter of ˜5-6 mm and thickness of ˜2 mm were formed and allowed to harden. The following inorganic substances were tested, in the form of powder: silica (Promeks Israel), hydroxyapatite (Biorad), calcium carbonate (Fluka).

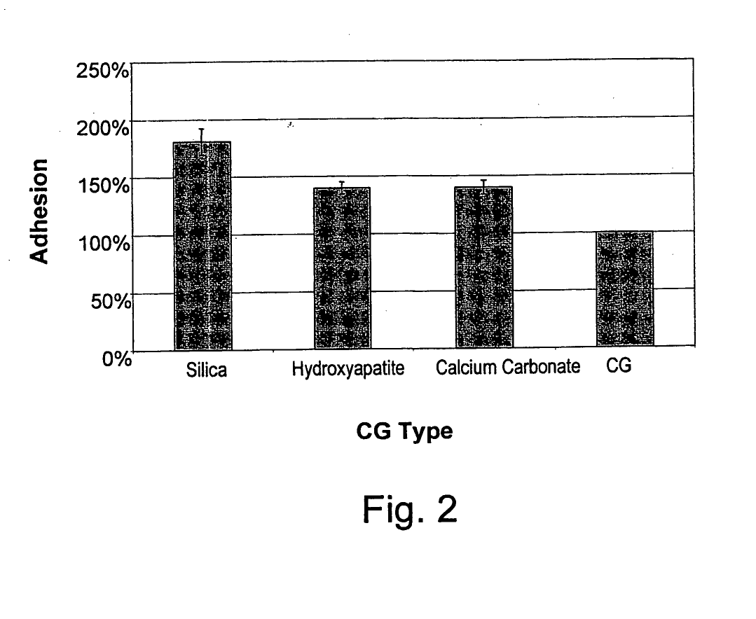

The discs were then incubated with the 3H-labeled bacteria, as described above. The results shown in FIG. 2 demonstrate that gum containing silica exhibited the best adhesive properties for Strep. mutans, 175% adhesion, while hydroxyapatite and calcium carbonate gave about 140% adhesion.

### Example 3

6.25 g of chewing gum base were heated in a microwave oven up to the paste state and mixed with 1.25 g of protein. Then chewing gum discs with a diameter of ˜5-6 mm and thickness of ˜2 mm were formed and allowed to harden. The following proteins were tested: gelatin type A (from porcine skin), gelatin type B (from bovine skin) and lectins (from wheat germ) [all from Sigma Chemical Co.].

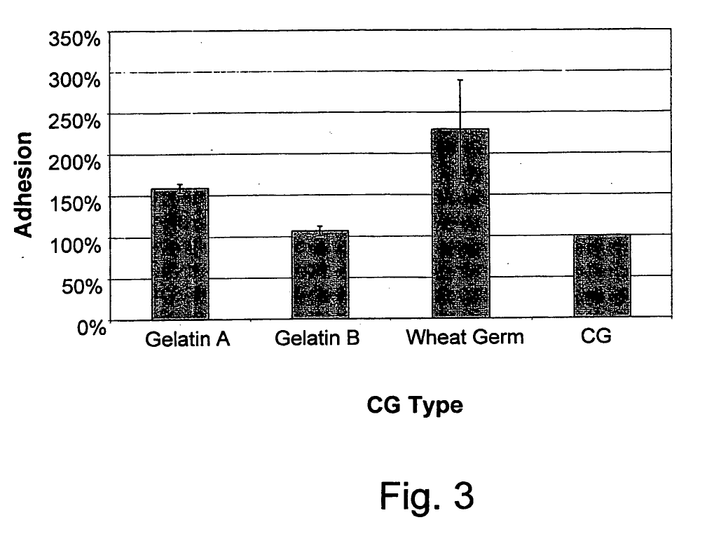

The discs were then incubated with the 3H-labeled bacteria, as described above. The results shown in FIG. 3 demonstrate that gum containing wheat germ (lectin) exhibited the best adhesive properties for Strep. mutans (230%), while Gelatin A gave 160%, compared to Gelatin B which was practically ineffective in adsorbing the same bacteria, compared to the chewing gum base (CG).

### Example 4

6.25 g of chewing gum base were heated in a microwave oven up to the paste state and mixed with 1.1 g of chitosan (Sigma), which is a polycation, together with 0.15 g of citric acid. Then chewing gum discs with diameter of ˜5-6 mm and thickness of ˜2 mm were formed and allowed to harden.

The discs were then incubated with the 3H-labeled bacteria, as described above.

It was found that the combination of chitosan with citric acid enhanced the adhesion effect (to 800%). As described in Example 1, chitosan without citric acid was less effective, 400% adhesion.

### Example 5

Gum discs were prepared as above, but adding two agents to the gum base. Thus, each disc contained gum base, and lower concentration of each additive, compared to those described in Example 1, 8.5% sodium alginate and 8.5% CMC.

The results show that the adhesion of Strep. mutans to the chewing gum which contained the two agents was 1100% as compared to CMC gum (450%), but lower than alginate alone (2000%).

### Example 6

Several chewing gum formulations were tested also with the following oral bacteria: Streptococcus sobrinus 6715; Streptococcus salivarius ATCC 25975; Actinomyces viscosus ATCC 43146. The results are described in Table 1.

TABLE 1

| | Strep. | Strep. | |
|---|---|---|---|
| | sobrinus | salivarius | A. viscosus |
| Chewing gum | | | |
| supplemented | | | |
| with | | | |
| Na Alginate | 420% | 370% | 490% |
| Chitosan | 80% | 150% | 110% |
| Xanthane | 210% | 125% | 105% |
| CMC | 190% | 295% | 210% |
| Hydroxyapatite | 150% | 210% | 350% |
| Silica | 50% | 50% | 190% |
| Dextran 190 | 40% | 50% | 80% |
| Ethyl-cellulose | 95% | 50% | 190% |
| Lecithin | 65% | 110% | 205% |
| Gelatin-A | 110% | 130% | 95% |
| Guar gum | 75% | 210% | 110% |
| Chewing gum | 100% | 100% | 100% |
| base | | | |

Similar to the results obtained in the experiments performed with Strep. mutans, also the other above oral bacteria showed enhanced adsorption to several chewing gum bases containing adhesion promoting additives. Here, too, the best additive was sodium alginate, followed by CMC, xanthane and hydroxyapatite. Other additives such as silica, dextran, ethylcellulose, lecithin, Gelatin-A and guar gum were ineffective in promoting adhesion.

### Example 7

This example shows adsorption of glucosyltransferase, an extra cellular enzyme associated with the oral biofilm.

Chewing gum discs were prepared as described in Example 1, using 17% sodium alginate as the active agent.

Cell free glucosyltransferase (GTF) was prepared as described by Steinberg et al. (23). Briefly. Strep. sobrinus bacteria were grown in dialysis tubes for 24 hours. Next, the bacteria were centrifuged and the supernatant fluid was collected. The cell free enzymes in the supernatant fluid were isolated and purified using ultrafiltration methods.

The sodium alginate containing chewing gum discs and control chewing gum discs (without alginate) were subjected to the GTF preparation for 35 minutes. Next, the samples were thoroughly washed and a GTF substrate containing sucrose 2% supplemented with radioactive labeled sucrose was introduced to the samples for 1.5 hour. After incubation the samples were washed and the amount of radioactivity was measured in a scintillation counter and expressed as counts per minutes (CPM) (Table 2).

TABLE 2

| | Chewing gum | |
|---|---|---|
| | base + Na | Chewing gum |
| | alginate | base |
| GTF activity | 2750 | 50 |
| (cpm) | | |

References
1. Albandar J. M., Rams T. E. Periodontol 2000. 2002; 29:7-10.
2. Mandel I. D. Compend Contin Educ Dent. 23:403406, 2000.
3. Steinberg, D., In: Handbook of Bacterial Adhesion: Principles, Methods, and Applications. Eds An and Friedman 2000; 353-370, 2000.
4. Liljemark, W. F., Bloomquist, C., Critical Reviews in Oral Biology and Medicine 7:180-198 1996.
5. Marshall, K. C. In “Microbial Adhesion and Aggregation”, K. C. Marshall, Ed., Springer-Verlag, Berlin, 1984, pp.1-3.
6. Robb, I. D. In “Microbial Adhesion and Aggregation”, K. C.Marshall, Ed., Springer-Verlag, Berlin, 1984, pp. 3949.
7. Dazzo, F. B. In “Microbial Adhesion and Aggregation” K. C. Marshall, Ed. Springer-Verlag, Berlin, 1984, pp. 85-93.
8. Rutter, P. R. et al. In “Microbial Adhesion and Aggregation”, K. C. Marshall, Ed., Springer-Verlag, Berlin, 1984, pp. 4-19.
9. Kjelleberg, J. In “Microbial Adhesion and Aggregation”, K. C. Marshall, Ed., Springer-Verlag, Berlin, 1984, pp. 51-70.
10.Jones, G. W. In “Microbial Adhesion and Aggregation”, K. C. Marshall, Ed., Springer-Verlag, Berlin, 1984, pp. 71-84.
11. Knowles, J., Roller, S., J. Food Prot. 64:1542-1548, 2001.
12.Francois, P., et al., Clin. Oral Implants Res. 8:217-225, 1997.
13. Stack, S., et al., Biochemistry 30:2073-2077, 1991.
14.Kelly, C. G., Younson, J. S. Expert Opin. Investig. Drugs. 9:1711-1721, 2000.
15.Marshall, K. C., “Interfaces in Microbial Ecology”, Haravrd University Press, Cambridge, 1976.
16.Veyries, M. L., et al., Antimicrob Agents Chemother. 44:1093-1096,2000.
17. McAllister, T. A., et al., J. Anim. Sci. 72:3004-3018,1994.
18.Berg, C. H., et al., Eur. J. Oral Sci. 109:316-324, 2001.
19. Wright, J. B., Michalopoulos, D. L. U.S. Pat. No. 5,593,599
20. Wright, J. B. U.S. Pat. No. 6,110,381
21.Wright, J. B., Johnson, M. C. U.S. Pat. No. 6,241,898
22. Donald, R. M., et al., U.S. Pat. No. 6,039,965.
23.Steinberg, D., et al., J. Dent. Res. 71:1797-1802 (1992).

1. An edible and/or chewable article of manufacture containing at least one food grade active substance having adsorption affinity towards at least one dental plaque (biofilm) constituent and capable of reducing and/or removing the oral biofilm while present in the mouth.

2. An article of manufacture according to claim 1, selected from the group consisting of chewing gums, sweets, candies, candy- and other nutritional bars, chocolates, ice creams, confectionery and bakery/pastry products, honey, dairy products and beverages.

3. An article of manufacture according to claim 1, selected from the group consisting of oral hygiene products such as tooth pastes, oral gels and mouthwashes.

4. An edible and/or chewable article of manufacture according to claim 1, being a chewing gum, wherein said chewing gum comprises a conventional gum base and at least one food grade active substance having adsorption affinity towards at least one dental plaque (biofilm) constituent and capable of reducing and/or removing the oral biofilm while present in the mouth.

5. A chewing gum according to claim 4, wherein said chewing gum has affinity towards said at least plaque constituent higher than that of said gum base.

6. A chewing gum according to claim 4, wherein the dental plaque (biofilm) constituent is a bacterium of the oral cavity of at least one species.

7. A chewing gum according to claim 6, wherein the bacterium is selected from the group consisting of Streptococcus mutans, Streptococcus sobrinus, Streptococcus salivarius and Actinomyces viscosus.

8. A chewing gum according to claim 4, wherein the dental plaque constituent is selected from the group consisting of proteins and bacterial cell-free enzymes.

9. A chewing gum according to claim 4, wherein said active substance is a polysaccharide or a non-toxic salt thereof.

10. A chewing gum according to claim 9, wherein the polysaccharide or a non-toxic salt thereof is selected from the group consisting of alginates, preferably sodium alginate, chitosan, carboxymethylcellulose, agar and carrageenan.

11. A chewing gum according to claim 4, wherein said active substance is an inorganic substance.

12. A chewing gum according to claim 11, wherein the inorganic material is selected from the group consisting of silica, hydroxyapatite and calcium carbonate.

13. A chewing gum according to claim 4, wherein said active substance is a protein.

14. A chewing gum according to claim 13, wherein the protein is selected from the group consisting of gelatins and lectins.

15. A chewing gum according to claim 4, wherein said active substance is a mixture of at least two substances selected from polysaccharides, proteins and inorganic substances.

16. A chewing gum according to claim 15, wherein said polysaccharide is selected from the group consisting of alginates, preferably sodium alginate, chitosan, carboxymethylcellulose, agar and carrageenan, said protein is selected from lectin and gelatin A, and said inorganic material is selected from the group consisting of silica, hydroxyapatite and calcium carbonate.

17. A chewing gum according to claim 4, for removing and/or for preventing or reducing dental plaque (biofilm).

18. A chewing gum according claim 4, further comprising at least one food grade additive, particularly coloring agents, flavoring agents, breath freshening agents, pH controlling agents and preserving agents.

19. A chewing gum according to claim 18, wherein said active substance is chitosan and said pH controlling agent is citric acid.

20. A chewing gum according to claim 4, in unit form, preferably in the form of a bar, drop, sphere or stick.

21. An article of manufacture according to claim 1, for removing and/or for preventing or reducing dental plaque (biofilm).

22. A method of treating a mouth comprising preparing an edible and/or chewable article of manufacture adapted for removing dental plaque (biofilm) and/or for preventing or reducing dental plaque that includes an active substance which has adsorption affinity towards dental plaque (biofilm) constituents, inserting said article of manufacture into a mouth, and exposing teeth in the mouth to the active substance.

23. Method according to claim 22, wherein said article of manufacture is selected from the group consisting of chewing gums, sweets, candies, candy- and other nutritional bars, ice creams, confectionery and bakery/pastry products, chocolates, honey, dairy products and beverages.

24. Method according to claim 22, wherein said article of manufacture is selected from the group consisting of oral hygiene products such as tooth pastes, oral gels and mouthwashes.

25. Method according to claim 22, wherein said article of manufacture is a chewing gum which contains a conventional chewing gum base and an active food grade substance which has adsorption affinity towards dental plaque (biofilm) constituents, wherein the adsorption affinity of said chewing gum towards dental plaque (biofilm) constituents is higher than the affinity of said gum base.

26. Method according claim 22, wherein the dental plaque (biofilm) constituent is a bacterium of the oral cavity of at least one species.

27. Method according to claim 26, wherein the bacterium is selected from the group consisting of Streptococcus mutans, Streptococcus sobrinus, Streptococcus salivarius and Actinomyces viscosus.

28. Method according to claim 22, wherein the dental plaque constituent is selected from the group consisting of proteins and bacterial cell-free enzymes.

29. Method according to claim 22, wherein said active substance is a polysaccharide or a non-toxic salt thereof, particularly a polysaccharide selected from the group consisting of alginates, preferably sodium alginate, chitosan, carboxymethylcellulose, agar and carrageenan.

30. Method according to claim 22, wherein said active substance is an inorganic substance, preferably an inorganic substance selected from the group consisting of silica, hydroxyapatite and calcium carbonate.

31. Method according to claim 22, wherein said active substance is a protein, particularly a protein selected from the group consisting of gelatins, particularly gelatin A, and lectins.

32. Method according claim 22, wherein said active is a mixture of at least two substances selected from polysaccharides, proteins and inorganic substances.

33. Method according to claim 32, wherein said polysaccharide is selected from the group consisting of alginates, preferably sodium alginate, chitosan, carboxymethylcellulose, agar, CMC and carrageenan, said protein is selected from lectins and gelatin A, and said inorganic material is selected from the group consisting of silica, hydroxyapatite and calcium carbonate.
